(19) United States
(12) Patent Application Publication
(10) Pub. No.: US 2010/0179162 A1
(43) Pub. Date: Jul. 15, 2010

(54) Crystal forms of 4-[6-methoxy-7(3-piperidin-1-yl-propoxy) quinazoline-4yl) piperazine-1-carboxylic acid (4-isopropoxyphenyl)-amide

(75) Inventors: Craig Grant, Burwell (GB); Hoa Q Luong, Newton, MA (US); Mark D. Angelino, Beverly, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals , Inc., Cambridge, MA (US)

(21) Appl. No.: 11/988,956

(22) PCT Filed: Jul. 20, 2006
(86) PCT No.: PCT/US2006/028231

§ 371 Date: Apr. 1, 2010

Related U.S. Application Data
(60) Provisional application No. 60/700,926, filed on Jul. 20, 2005.

(51) Int. Cl. A61K 31/496; C07D 403/14; A61P 35/00; A61P 35/02

(52) U.S. Cl. 514/252.17; 544/283

(57) ABSTRACT

Crystalline forms of the sulfate salt of 4-[6-methoxy-7-(3-piperidin-1-yl-propoxy)quinazolin-4-yl]piperazine-1-carboxylic acid (4-isopropoxyphenyl)-amide, which may be used in pharmaceutical applications, are disclosed. Particular single crystalline forms of the sulfate salt are characterized by a variety of properties and physical measurements. As well, methods of producing the sulfate salts, and using such salts to inhibit excessive tyrosine kinase activity in subjects to treat a number of diseases including cardiovascular disease (e.g., arteriosclerosis and vascular reobstruction), cancer (e.g., leukemia such as acute lymphocytic leukemia), glomerulosclerosis fibrotic diseases and inflammation, and general treatment of cell-proliferative diseases, are also discussed.

### RELATED APPLICATION(S)

This application claims the benefit of U.S. Provisional Application No. 60/700,926, filed on Jul. 20, 2005. The entire teachings of the above application are incorporated herein by reference.

### BACKGROUND OF THE INVENTION

The compound 4-[6-methoxy-7-(3-piperidin-1-yl-propoxy)quinazolin-4-yl]piperazine-1-carboxylic acid (4-isopropoxyphenyl)-amide (herein the “Compound”) is a novel small molecule drug candidate that inhibits the FLT-3 receptor tyrosine kinase (RTK). Approximately 30% of patients with acute myeloid leukemia (AML) have a mutation in their FLT-3 gene, specifically an internal tandem duplication (ITD), which may be implicated in the growth and survival of the leukemia. Cancer Cell (2002), 1(5), 421-432.

In preclinical studies, the Compound selectively killed human FLT-3/ITD-positive AML cancer cells. Additionally, because the Compound targets the tyrosine kinases associated with the platelet-derived growth factor receptor (PDGFR) and c-KIT, as well as FLT-3, it may have broader potential utility for other hematologic malignancies or solid tumors. The Compound is currently in clinical trials as a potential treatment for AML. The use of the Compound to treat AML, was granted fast-track status by the U.S. Food and Drug Administration (FDA). See www.m1nm.com, under R&D and Pipeline.

The Compound is described in Pandey et al., in J. Med. Chem. (2002), 45, 3772-3793. In this reported synthesis, the Compound is isolated as the hydrochloride salt after being dried in vacuo. One disadvantage of the method is that the hydrochloride salt so formed does not have ideal stability, mainly due to its hygroscopicity. WO 02/36587 describes a method for making the Compound as a sulfate salt. However, the method results in a mixture of solid forms. Measurements taken by X-ray powder diffraction on samples prepared in accordance with WO 02/36587 show the presence of more than one solid form of the sulfate salt of the Compound.

Polymorphism refers to the ability of a chemical compound to exist in two or more crystalline forms that have different properties. Chemical polymorphs have identical chemical structures, but different properties. Key physical properties that may be affected depending on the polymorph include hygroscopicity, solubility, storage stability, density, hardness, flow properties and bioavailability.

Optimal physical and chemical properties of a drug candidate are desired in order to select the best candidate for therapeutic treatment. Moreover, in order for a drug product to be suitable for human therapeutic use under the Food and Drug Administration regulations, certain physical properties of the drug must be favorable and capable of stringent quality control.

Polymorphs may be characterized by their X-ray powder diffraction patterns. Other useful analytical techniques for characterizing polymorphs include differential scanning calorimetry (DSC), and thermogravimetric analysis (TGA). There are many examples of new solid forms that have been reported for previously known pharmaceutical products. See, for example, Journal of Pharmaceutical and Biomedical Analysis, 3(4), pp 303-313 (1985) and U.S. Pat. Nos. 5,945,405; 6,391,906; 6,376,469 and 6,514,953.

Investigations of polymorphs of drugs may be undertaken by trial and error which involves running a series of, for example, recrystallizations of the drug using a range of different re-crystallization conditions and then analyzing the re-crystallized products. The trial and error approach is tedious and time-consuming. Alternatively, the discovery of new polymorphs may be done in an automated fashion using an apparatus specially designed to investigate different physical and/or chemical forms of materials (see, for example, WO 01/77690).

### SUMMARY OF THE INVENTION

It has been found that the sulfate salt of the compound can be crystallized under well-defined conditions to provide certain preferred solid crystal forms. These crystalline forms are homogeneous and have superior physical properties compared to the properties associated with the prior art forms of the Compound.

One embodiment of the invention is directed toward a sulfate salt of 4-[6-methoxy-7-(3-piperidin-1-yl-propoxy)quinazolin-4-yl]piperazine-1-carboxylic acid (4-isopropoxyphenyl)-amide, which is at least 60% by weight crystalline. More particularly, the sulfate salt is 60%, 80%, or 95% by weight a single crystalline form, the possible single crystalline forms being described herein.

In another embodiment of the invention, a pharmaceutical composition comprises a pharmaceutically acceptable carrier or diluent; and a sulfate salt of 4-[6-methoxy-7-(3-piperidin-1-yl-propoxy)quinazolin-4-yl]piperazine-1-carboxylic acid (4-isopropoxyphenyl)-amide. The sulfate salt is at least 60% by weight crystalline. More particularly, the sulfate salt of the pharmaceutical composition is 60%, 80%, or 95% by weight a single crystalline form, the possible single crystalline forms being described herein.

Embodiments of the invention are also directed toward a method of treating a subject in need of tyrosine kinase inhibition, a subject with cancer, or a subject with leukemia by administering an effective amount of a sulfate salt of 4-[6-methoxy-7-(3-piperidin-1-yl-propoxy)quinazolin-4-yl]piperazine-1-carboxylic acid (4-isopropoxyphenyl)-amide, which is at least 60% by weight in a single crystalline form, to a subject in need thereof, the possible single crystalline forms being described herein.

Embodiments of the invention are also directed to methods of preparing a sulfate salt of 4-[6-methoxy-7-(3-piperidin-1-yl-propoxy)quinazolin-4-yl]piperazine-1-carboxylic acid (4-isopropoxyphenyl)-amide. The method comprises providing a pharmaceutical composition of the sulfate salt in a single crystalline form and granulating the composition.

### BRIEF DESCRIPTION OF THE DRAWINGS

FIG. 1 depicts a XRPD pattern from measuring a sample of Form C of the Sulfate Salt, consistent with an embodiment of the invention.

FIG. 2 depicts DSC and TGA results from measuring a sample of Form C of the Sulfate Salt, consistent with an embodiment of the invention.

FIG. 3 depicts moisture isotherms from exposing a sample of Form C of the Sulfate Salt to varying relative humidities at 25° C., in accord with an embodiment of the invention.

FIG. 4 depicts a XRPD pattern from measuring a sample of Form B of the Sulfate Salt, consistent with an embodiment of the invention.

FIG. 5 depicts DSC and TGA results from measuring a sample of Form B of the Sulfate Salt, consistent with an embodiment of the invention.

FIG. 6 depicts a XRPD pattern from measuring a sample of Form F of the Sulfate Salt, consistent with an embodiment of the invention.

FIG. 7 depicts DSC and TGA results from measuring a sample of Form F of the Sulfate Salt, consistent with an embodiment of the invention.

FIG. 8 depicts a XRPD pattern from measuring a sample of Form G of the Sulfate Salt at 100° C., consistent with an embodiment of the invention.

FIG. 9 depicts XRPD intensities as a function of 20 and varying temperatures for a sample of Form G of the Sulfate Salt, in accord with an embodiment of the invention.

FIG. 10 depicts a XRPD pattern from measuring a sample of Form H of the Sulfate Salt, consistent with an embodiment of the invention.

FIG. 11 depicts DSC and TGA results from measuring a sample of Form H of the Sulfate Salt, consistent with an embodiment of the invention.

FIG. 12 depicts a XRPD pattern from measuring a sample of Form D of the Sulfate Salt, consistent with an embodiment of the invention.

FIG. 13 depicts DSC and TGA results from measuring a sample of Form D of the Sulfate Salt, consistent with an embodiment of the invention.

FIG. 14 depicts moisture isotherms from exposing a sample of Form D of the Sulfate Salt to varying relative humidities at 25° C., in accord with an embodiment of the invention.

### DETAILED DESCRIPTION OF THE INVENTION

Embodiments of the present invention provide new solid forms, or polymorphs, of the compound 4-[6-methoxy-7-(3-piperidin-1-yl-propoxy)quinazolin-4-yl]piperazine-1-carboxylic acid (4-isopropoxyphenyl)-amide. The chemical structure of the Compound is given by formula (I):

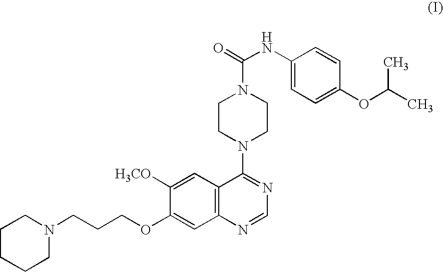

A particular embodiment of the invention is directed toward a sulfate salt of 4-[6-methoxy-7-(3-piperidin-1-yl-propoxy)quinazolin-4-yl]piperazine-1-carboxylic acid (4-isopropoxyphenyl)-amide (herein the “Sulfate Salt”) that is crystalline. More particularly, the crystalline Sulfate Salt may be a single crystalline form. The crystalline Sulfate Salt, or single crystalline form of the Sulfate Salt, may be at least a particular percent by weight of a total amount of the Sulfate Salt. Particular percentages include 60%, 65%, 70%, 75%, 80%, 85%, 86%, 87%, 88%, 89%, 90%, 91%, 92%, 93%, 94%, 95%, 96%, 97%, 98%, 99%, 99.5%, 99.9%, or any percentage between 60% and 100%.

As used herein, “crystalline” refers to a solid having a highly regular chemical structure. In particular, a crystalline Sulfate Salt may be produced as one or more single crystalline forms of the Sulfate Salt. A single crystalline form of the Sulfate Salt may include a single crystal or a plurality of crystals in which each crystal has the same crystal form (e.g., all the crystals are Form C). As well, the terms “single crystalline form” and “polymorph” are synonymous; the terms distinguish between crystals that have different properties (e.g., different XRPD patterns, different DSC scan results). Pseudopolymorphs are typically different solvates of a material, and thus their properties differ from one another. Thus, the term “polymorph” as it is used herein includes alternate crystal forms of the Sulfate Salt as well as pseudopolymorphs (e.g., solvated crystalline forms of the Sulfate Salt).

A crystalline Sulfate Salts can be a single crystalline form of the Sulfate Salt (e.g., Form C), or a mixture of different single crystalline forms (e.g., a mixture of Forms C and B, a mixture of any combination of Forms C, B, and F).

Embodiments of the invention are also directed to a crystalline Sulfate Salt, wherein at least a particular percentage by weight of the crystalline Sulfate Salt is a specific single crystalline form. Particular weight percentages may be 60%, 65%, 70%, 75%, 80%, 85%, 86%, 87%, 88%, 89%, 90%, 91%, 92%, 93%, 94%, 95%, 96%, 97%, 98%, 99%, 99.5%, 99.9%, or any percentage between 60% and 100%.

When a particular percentage by weight of a Sulfate Salt is a single crystalline form, the remainder of the Sulfate Salt is some combination of amorphous form of the Sulfate Salt, and/or one or more crystalline forms of the Sulfate Salt excluding the single crystalline form. When the crystalline Sulfate Salt is defined as one or more particular forms of the Sulfate Salt, the remainder is made up of amorphous form and/or crystalline forms other than the one or more particular forms that are specified. Examples of a single crystalline form include Forms B, C, D, F, G, and H of the Sulfate Salt, as well as descriptions of a single crystalline form characterized by one or more properties as discussed herein.

In the following description of particular polymorphs of the sulfate salt of 4-[6-methoxy-7-(3-piperidin-1-yl-propoxy)quinazolin-4-yl]piperazine-1-carboxylic acid (4-isopropoxyphenyl)-amide, embodiments of the invention may be described with reference to a particular crystalline “Form” of the Sulfate Salt (e.g., Form C). However, the particular crystalline forms of the Sulfate Salt may also be characterized by one or more of the characteristics of the polymorph as described herein, with or without regard to referencing a particular “Form”.

### Form C

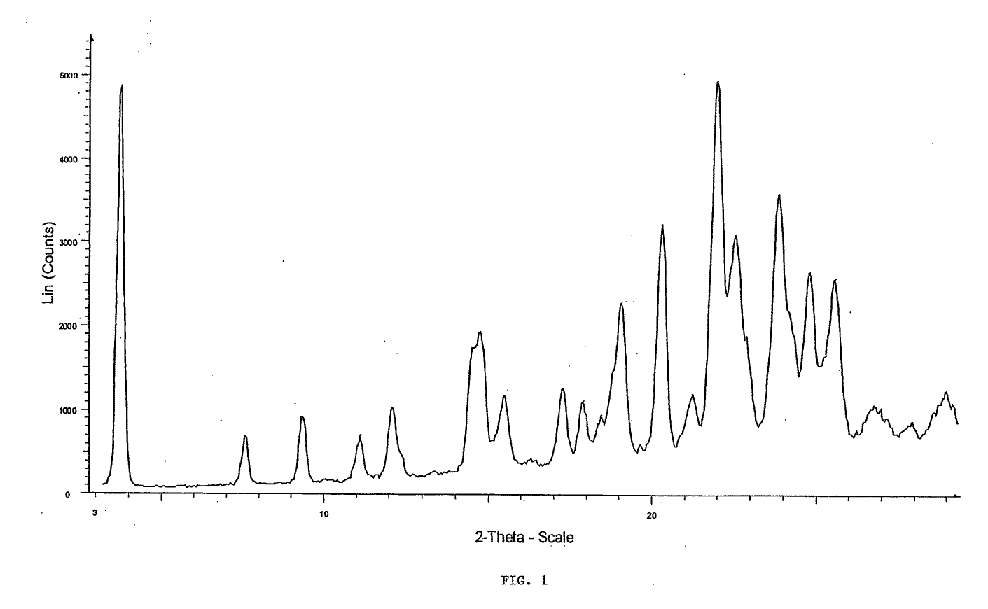

In one embodiment of the invention, a single crystalline form of the Sulfate Salt is characterized as Form C. This polymorph is also characterized by the X-ray powder diffraction (herein referred to as “XRPD”) pattern shown in FIG. 1, obtained using CuKα radiation. In a particular embodiment of the invention, the polymorph is characterized by one or more of the peaks of FIG. 1, as listed in Table 1. In more particular embodiments of the invention, the polymorph is characterized by one, two, three, four, five, six, seven, eight, or nine of the peaks in Table 1; even more particularly, the peaks are identified at 2θ angles of 3.7°, 11.1°, 12.1°, 15.5°, 17.3°, 22.6°, 23.9°, 25.6°, and 29.0°. All 2θ angles are known to within an error of ±0.2°.

TABLE 1
XRPD Peaks from FIG. 1

| 2θ angle (°) | d value (Å) | Intensity % |
|---|---|---|
| 3.7 | 23.8 | 98.0 |
| 7.6 | 11.7 | 14.0 |
| 9.3 | 9.5 | 18.4 |
| 11.1 | 8.0 | 13.6 |
| 12.1 | 7.3 | 20.5 |
| 13.3 | 6.6 | 5.5 |
| 14.7 | 6.0 | 39.4 |
| 15.5 | 5.7 | 23.5 |
| 17.3 | 5.1 | 25.5 |
| 17.9 | 5.0 | 22.1 |
| 18.4 | 4.8 | 19.0 |
| 19.1 | 4.6 | 46.0 |
| 20.3 | 4.4 | 64.8 |
| 21.2 | 4.2 | 24.3 |
| 22.0 | 4.0 | 100 |
| 22.6 | 3.9 | 62.5 |
| 23.9 | 3.7 | 72.3 |
| 24.8 | 3.6 | 53.4 |
| 25.6 | 3.5 | 51.7 |
| 26.8 | 3.3 | 21.6 |
| 27.9 | 3.2 | 18.1 |
| 29.0 | 3.1 | 25.0 |

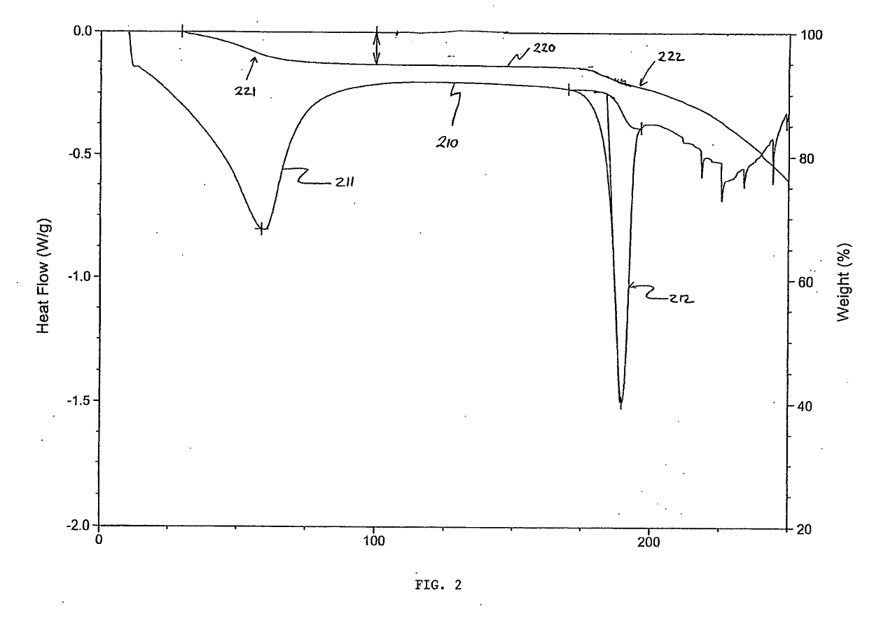

In another embodiment of the invention, Form C of the Sulfate Salt is characterized by the differential scanning calorimetry (herein referred to as “DSC”) profile 210 shown in FIG. 2. The profile 210 plots the heat flow as a function of temperature from a sample containing Form C. The profile 210 is characterized by two endothermic transitions 211, 212. The first transition 211 exhibits a maximum heat flow at 59° C. The second transition 212 exhibits a maximum heat flow at 190° C., and begins at a temperature of about 184° C. The integrated heat loss of this transition 212 is calculated to be 49.6 J/g of sample. These temperatures have an error of ±3° C., and are conducted at a temperature scanning rate of 10° C./minute.

Form C is also characterized by the thermal gravimetric analysis (herein referred to as “TGA”) profile 220 shown in FIG. 2. The profile 220 graphs the percent loss of weight of the sample as a function of temperature, the temperature rate change being about 10° C./min. The weight loss 221 represents a loss of about 5.2% of the weight of the sample as the temperature is changed from room temperature to 100° C. Another weight loss 222 begins at about the same temperature as the beginning of the second endothermic transition 212 of the DSC profile 210. This weight loss 222 corresponds with the melting and/or decomposition of the sample. Thus, the sample is characterized by having a melting point/decomposition temperature of about 184° C. These temperatures have an error of ±3° C.

Form C is also characterized by having a hydration of about 1.8 to about 2.0 water molecules per molecule of Sulfate Salt. The first weight loss 221 of the TGA profile 220 corresponds to a loss of about 2.0 water molecules per molecule of Sulfate Salt, if the weight loss is attributed only to the complete dehydration of the form of the Sulfate Salt. The hydration of Form C is confirmed independently by measurements on samples of the Sulfate Salt using Karl Fischer titration.

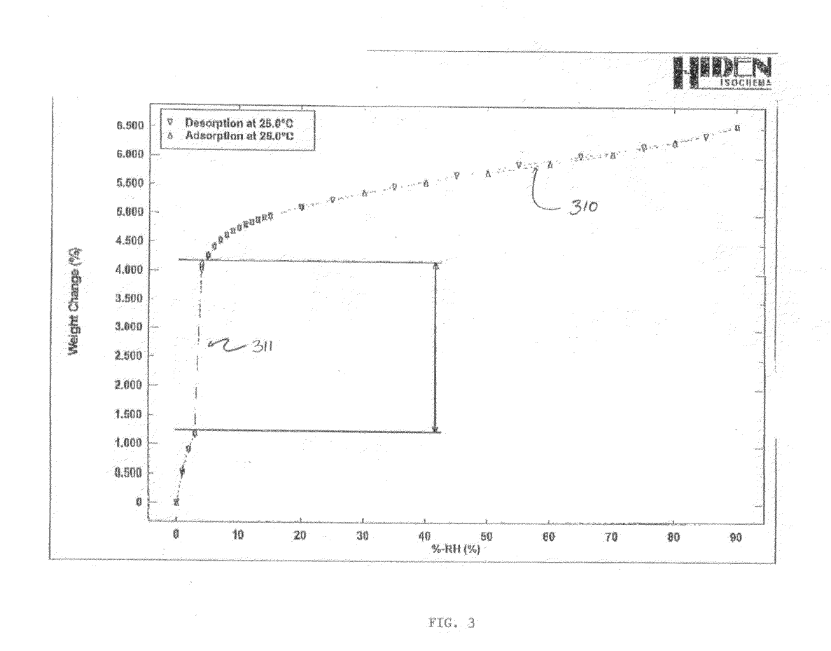

Another embodiment of the invention utilizes the vapor sorption profiles 310, as shown in FIG. 3, to characterize a sample of Form C of the Sulfate Salt. The profiles 310 show the change in weight of a sample of Form C, on an anhydrous basis, as the relative humidity of the environment is changed between 0% and 90% at a temperature of 25° C. The profiles 310 superimpose each other. Since the starting and ending points of the profiles 310 superimpose, Form C is characterized by reversible sorption/desorption phenomena. Also, the profiles 310 show a step change 311 in weight at a relative humidity of about 3% or 4%. This step change 311 is characterized by a weight change of about 3.0% on an anhydrous basis of sample. Thus, Form C is characterized as a hydrated polymorph that transforms to one or more other forms when the relative humidity is dropped below about 3% or 4% at 25° C. This transformation also indicates that Form C contains approximately 1.8 to 2.0 waters of hydration per molecule of the Compound.

### Form B

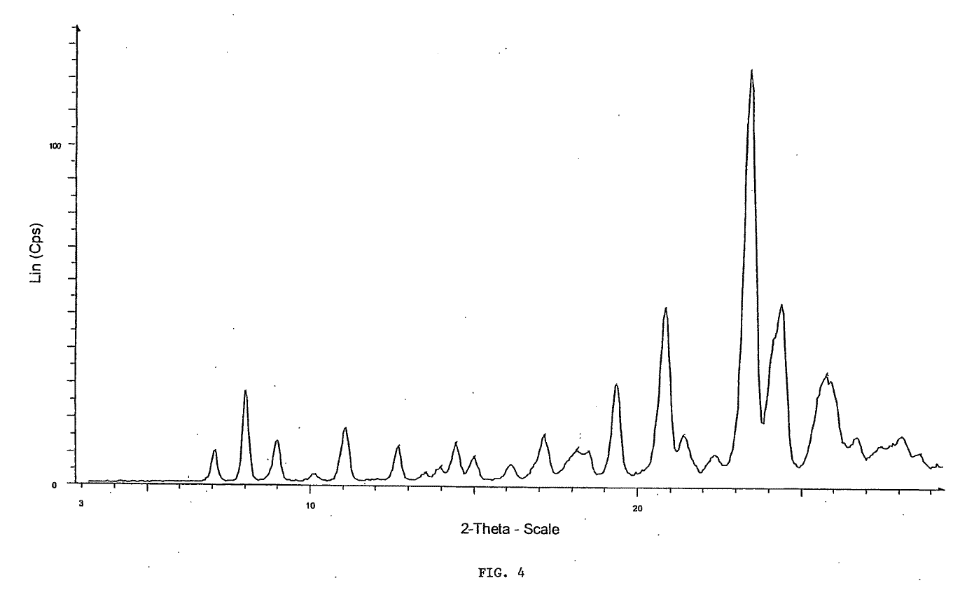

In an embodiment of the invention, a single crystalline form of the Sulfate Salt is characterized as Form B. The polymorph is also characterized by the X-ray powder diffraction (XRPD) pattern shown in FIG. 4, using CuKα radiation. In a particular embodiment of the invention, the polymorph is characterized by one or more of the peaks listed in Table 2; the peaks corresponding with particular peaks in FIG. 4. In more particular embodiments of the invention, the polymorph is characterized by one, two, three, four, five, six, seven, eight, nine, ten, eleven, twelve, or thirteen of the peaks in Table 2; even more particularly, the peaks are identified at 2θ angles of 7.1°, 8.0°, 9.0°, 11.1°, 12.7°, 14.4°, 15.0°, 16.1°, 17.1°, 19.4°, 20.9°, 23.4°, and 24.4°. All 2θ angles are known to within an error of ±0.2°.

TABLE 2
XRPD Peaks from FIG. 4

| 2θ angle (°) | d value (Å) | Intensity % |
|---|---|---|
| 7.1 | 12.5 | 8.1 |
| 8.0 | 11.0 | 22.2 |
| 9.0 | 9.9 | 10.6 |
| 10.1 | 8.7 | 2.5 |
| 11.1 | 8.0 | 14.0 |
| 12.7 | 7.0 | 9.2 |
| 13.5 | 6.5 | 3.0 |
| 14.0 | 6.3 | 4.3 |
| 14.4 | 6.1 | 10.2 |
| 15.0 | 5.9 | 7.1 |
| 16.1 | 5.5 | 5.2 |
| 17.1 | 5.2 | 12.2 |
| 18.5 | 4.8 | 8.5 |
| 19.4 | 4.6 | 24.6 |
| 20.9 | 4.3 | 42.8 |
| 21.4 | 4.1 | 12.3 |
| 22.4 | 4.0 | 7.9 |
| 23.4 | 3.8 | 100 |
| 24.2 | 3.7 | 36.0 |
| 24.4 | 3.6 | 43.7 |
| 26.7 | 3.3 | 12.2 |
| 27.5 | 3.2 | 10.2 |
| 28.1 | 3.2 | 12.4 |
| 28.7 | 3.1 | 8.4 |
| 29.2 | 3.1 | 6.1 |
| 25.9 | 3.4 | 25.5 |
| 25.8 | 3.5 | 26.5 |
| 18.1 | 4.9 | 8.7 |

In a related embodiment of the invention, Form B of the Sulfate Salt is characterized by one or more unit cell parameters. The cell parameters were derived from XRPD data taken on a sample of Form B at 120° K. The cell parameters are summarized in Table 3. The crystal is characterized by a Triclinic P-1 structure. Accordingly with this crystal structure, the crystal is characterized by having about 6 to about 7 lattice water molecule sites present for each drug molecule location. The actual number of water molecules present, however, depends upon the environmental conditions to which the crystal is exposed.

TABLE 3
Cell Parameters from XRPD Scan of Form B

a = 11.3542(3) Å
b = 12.8027(3) Å
c = 13.7349(4) Å
α = 76.1930(10)°
β = 71.7880(10)°
γ = 82.3110(10)°
V = 1837.95(8) Å³

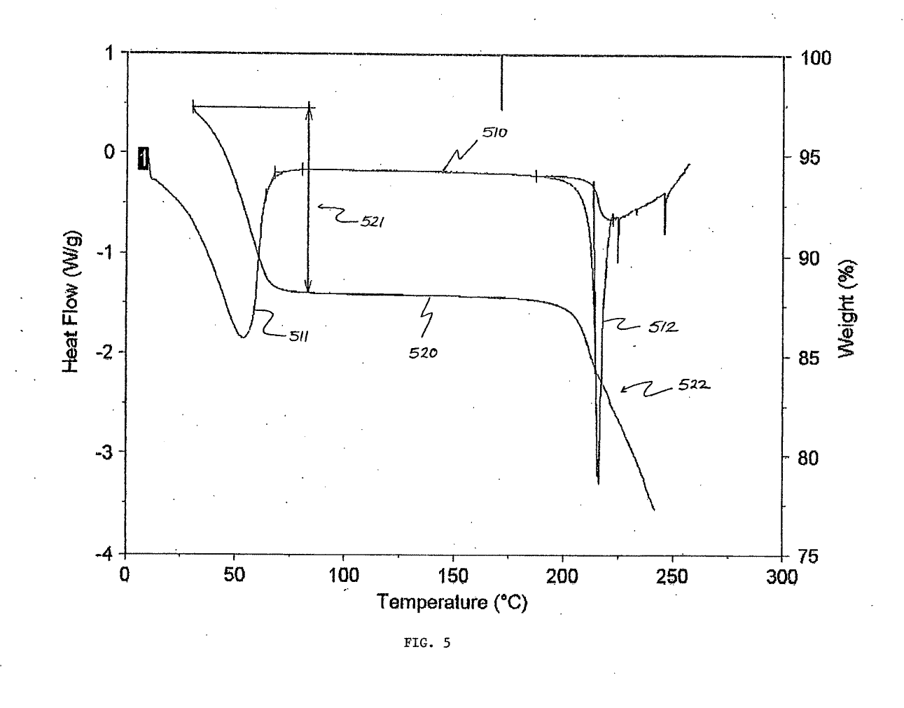

In another embodiment of the invention, Form B of the Sulfate Salt is characterized by the DSC profile 510 shown in FIG. 5. The graph 510 plots the heat flow as a function of temperature from a sample containing Form B, the temperature rate change being about 10° C./min. The profile 510 is characterized by two endothermic transitions 511, 512. The first transition 511 exhibits a maximum heat flow at 50° C. The second transition 512, corresponding with the melting point/decomposition of the sample, exhibits a maximum heat flow at about 216° C., and begins at a temperature of about 213° C. The integrated heat loss of this transition 512 is calculated to be 82.3 J/g of sample. These temperatures have an error of 3° C.

The polymorph is also characterized by the TGA profile 520 shown in FIG. 5. The profile 520 graphs the percent loss of weight of the sample as a function of temperature, the temperature rate change being about 10° C./min. The weight loss 521 represents a loss of about 9.3% of the weight of the sample as the temperature is changed from room temperature to 68° C. If the weight loss 521 is attributed solely to water loss, the weight loss corresponds to about 3.7 water molecules lost per drug molecule. This hydration loss of Form B in this state is confirmed by measurements on samples of the Sulfate Salt using Karl Fischer titration.

Another weight loss 522 begins at about the same temperature as the beginning of the second endothermic transition 512 of the DSC profile 510. This weight loss 522 corresponds with the melting and/or decomposition of the sample. Thus, the sample is characterized by having a melting point/decomposition temperature of about 213° C. These temperatures have an error of ±3° C.

### Form F

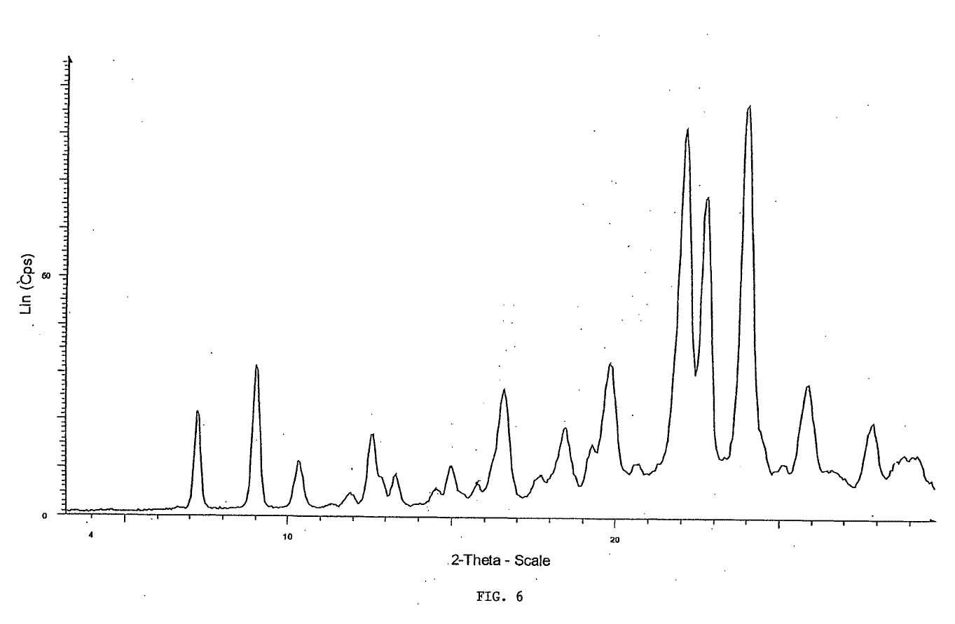

In another embodiment of the invention, a single crystalline form of the Sulfate Salt is characterized as Form F. The single crystalline form is also characterized by the characteristics of the X-ray powder diffraction (XRPD) pattern shown in FIG. 6, obtained using CuKα radiation. In a particular embodiment of the invention, the polymorph is characterized by one or more of the peaks in Table 4, the peaks being obtained from FIG. 6. In more particular embodiments of the invention, the polymorph is characterized by one, two, three, four, five, six, seven, eight, nine, or ten of the major peaks in Table 4; even more particularly, the peaks are identified at 2θ angles of 7.2°, 9.0°, 10.3°, 16.6°, 22.1°, 22.8°, 24.0°, 25.9°, 26.6°, and 27.9°. All 2θ angles are known to within an error of ±0.2°.

TABLE 4
XRPD Peaks from FIG. 6

| 2θ angle (°) | d value (Å) | Intensity % |
|---|---|---|
| 7.2 | 12.2 | 25.3 |
| 9.0 | 9.8 | 36.5 |
| 10.3 | 8.6 | 12.9 |
| 11.3 | 7.8 | 2.8 |
| 11.9 | 7.4 | 5.7 |
| 12.6 | 7.0 | 19.3 |
| 13.3 | 6.7 | 9.9 |
| 14.5 | 6.1 | 6.5 |
| 15.0 | 5.9 | 11.8 |
| 15.8 | 5.6 | 7.7 |
| 16.6 | 5.3 | 30.8 |
| 17.7 | 5.0 | 10.0 |
| 18.5 | 4.8 | 21.6 |
| 19.3 | 4.6 | 17.3 |
| 19.8 | 4.5 | 37.1 |
| 20.7 | 4.3 | 12.8 |
| 22.1 | 4.0 | 94.0 |
| 22.8 | 3.9 | 77.4 |
| 24.0 | 3.7 | 100 |
| 25.1 | 3.5 | 12.8 |
| 25.9 | 3.4 | 32.1 |
| 26.6 | 3.3 | 11.8 |
| 27.9 | 3.2 | 22.9 |
| 28.8 | 3.1 | 15.3 |

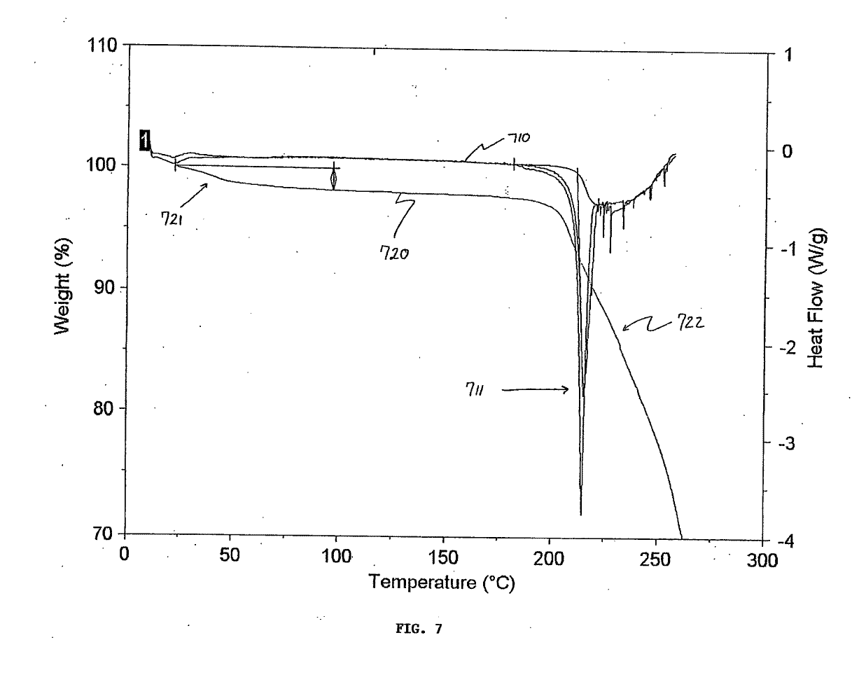

In another embodiment of the invention, Form F of the Sulfate Salt is characterized by the DSC profile 710 shown in FIG. 7. The graph 710 plots the heat flow as a function of temperature from a sample containing Form F, the sample being subjected to a temperature scanning rate of 10° C./min. The graph 710 is characterized by an endothermic transitions 711 exhibiting a maximum heat flow at about 216° C., and beginning at a temperature of about 212° C. The integrated heat loss of this transition 711 is calculated to be 81.5 J/g of sample. These temperatures have an error of ±3° C.

Form F is also characterized by the TGA profile 720 shown in FIG. 7. The graph 720 tracks the percent loss of weight of the sample as a function of temperature, the temperature rate change being 10° C./min. The weight loss 721 represents a loss of about 1.8% of the weight of the sample as the temperature is changed from room temperature to about 100° C. Another weight loss 722 begins at about the same temperature as the beginning of the endothermic transition 712 of the DSC profile 710. The weight loss 722 corresponds with the melting and/or decomposition of the sample. Thus, the sample is characterized by a melting point/decomposition temperature of about 212° C. These temperatures have an error of ±3° C.

Form F is also characterized by having a hydration of about 0.3 to about 0.7 water molecules per molecule of Sulfate Salt. The weight loss 721 of the TGA profile 720 corresponds to a loss of about 0.7 water molecules per molecule of Sulfate Salt, if the weight loss is attributed only to the complete dehydration of the form of the Sulfate Salt. The hydration of Form F is confirmed independently by measurements on samples of the Sulfate Salt using Karl Fischer titration, showing weight losses of about 1.64 weight percent and about 1.73 weight percent.

### Form G

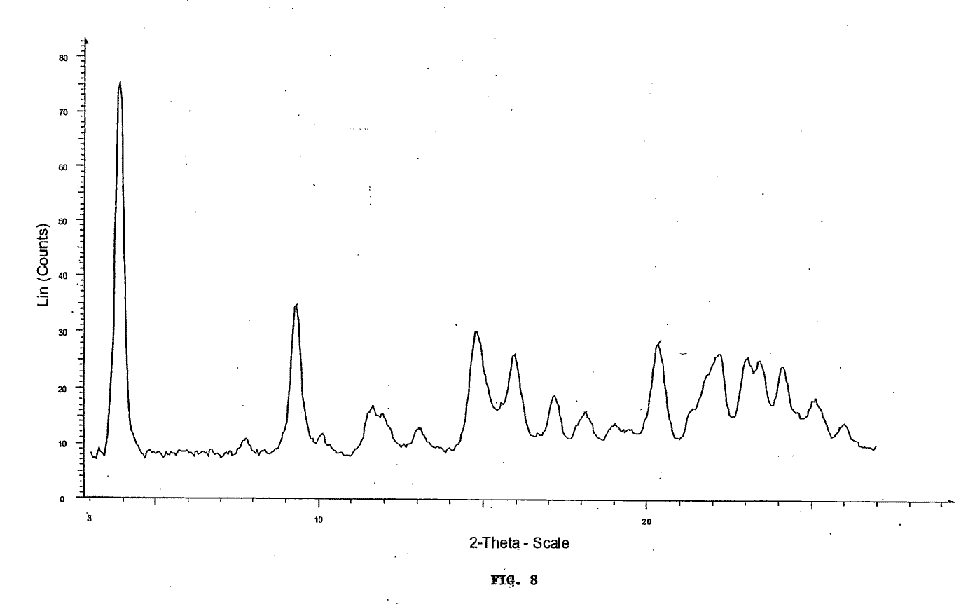

In another embodiment of the invention, a single crystalline form of the Sulfate Salt is characterized as Form G. The polymorph is characterized by the XRPD pattern shown in FIG. 8, derived from measuring a sample of Form G using CuKα radiation. In a particular embodiment of the invention, the polymorph is characterized by one, two, three, four, five, six, seven, eight, nine, or ten of the peaks in Table 5, the peaks being obtained from FIG. 8. More particularly, the peaks are chosen from 3.8°, 10.1°, 11.6°, 13.0°, 15.9°, 17.2°, 18.1°, 22.2°, 23.1°, and 23.4°. The 2θ angles have an error of ±0.2°.

TABLE 5
XRPD Peaks from FIG. 8

| 2θ angle (°) | d value (Å) | Intensity % |
|---|---|---|
| 3.8 | 23.0 | 100 |
| 7.7 | 11.4 | 14.5 |
| 9.3 | 9.5 | 45.6 |
| 10.1 | 8.8 | 15.6 |
| 11.6 | 7.6 | 22.6 |
| 13.0 | 6.8 | 17.0 |
| 14.8 | 6.0 | 40.2 |
| 15.9 | 5.6 | 34.8 |
| 17.2 | 5.2 | 25.2 |
| 18.1 | 4.9 | 20.9 |
| 19.0 | 4.7 | 18.1 |
| 20.4 | 4.4 | 37.4 |
| 21.4 | 4.1 | 22.3 |
| 22.2 | 4.0 | 35.1 |
| 23.1 | 3.8 | 34.0 |
| 23.4 | 3.8 | 33.5 |
| 24.1 | 3.7 | 32.6 |
| 25.1 | 3.5 | 24.7 |
| 26.0 | 3.4 | 18.6 |

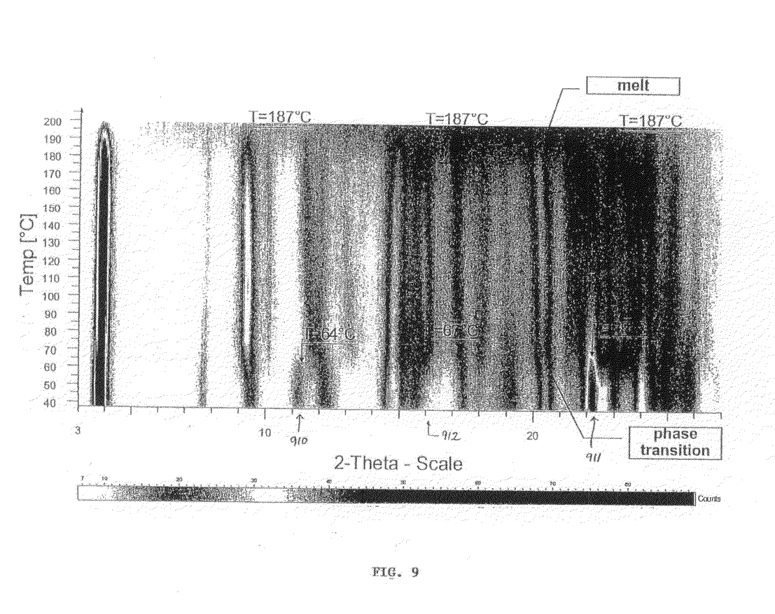

Form G is also characterized by the temperature-dependent XRPD data presented in FIG. 9. The contrast at any particular point in the two-dimensional area of FIG. 9 corresponds with an intensity reading associated with the corresponding 2θ angle and temperature at which the sample is examined. As shown in FIG. 9, the disappearance of the line 910, the fading of line 911, and the emergence of line 912 at about 64° C. to about 69° C. indicates the emergence of Form G at temperatures above about 65° C.

Form G of the Sulfate Salt is characterized as being anhydrous. Scans made using DSC and TGA confirm the dehydrated nature of Form G above a temperature of about 65° C.

### Form H

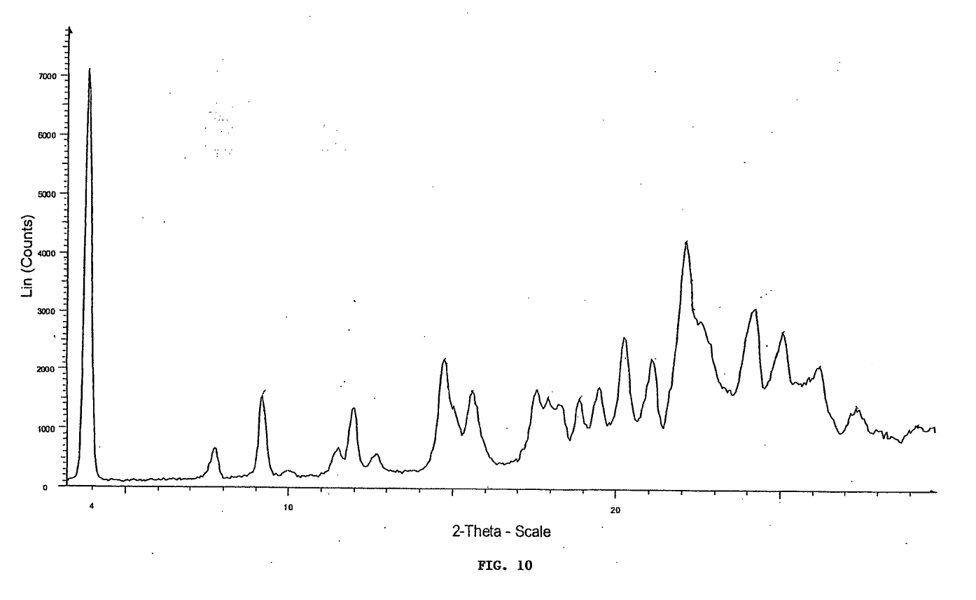

An embodiment of the invention is directed toward a single crystalline form of the Sulfate Salt designated Form H. A XRPD pattern created using CuKα radiation, characterizing the single crystalline form, is presented in FIG. 10. The polymorph is also characterized by one, two, three, four, five, six, seven, eight, nine, ten, or eleven of the peaks in FIG. 10, some of which are listed in Table 6. More particularly, the peaks are chosen from 3.8°, 11.5°, 12.0°, 12.7°, 15.6°, 17.6°, 18.3°, 19.5°, 24.2°, 25.1°, and 26.2°. The 2θ angles have an error of ±0.2°.

TABLE 6
XRPD Peaks from FIG. 10

| 2θ angle (°) | d value (Å) | Intensity % |
|---|---|---|
| 3.8 | 23.0 | 100 |
| 7.7 | 11.5 | 9.0 |
| 9.2 | 9.6 | 21.5 |
| 10.0 | 8.9 | 3.8 |
| 11.5 | 7.7 | 9.4 |
| 12.0 | 7.4 | 18.9 |
| 12.7 | 7.0 | 8.2 |
| 14.7 | 6.0 | 30.9 |
| 15.1 | 5.9 | 19.4 |
| 15.6 | 5.7 | 22.8 |
| 17.6 | 5.0 | 23.7 |
| 17.9 | 4.9 | 21.6 |
| 18.3 | 4.8 | 20.3 |
| 18.9 | 4.7 | 21.4 |
| 19.5 | 4.5 | 24.3 |
| 20.3 | 4.4 | 36.5 |
| 21.1 | 4.2 | 31.4 |
| 22.1 | 4.0 | 59.3 |
| 22.6 | 3.9 | 40.3 |
| 24.2 | 3.7 | 43.5 |
| 24.9 | 3.6 | 31.8 |
| 25.1 | 3.6 | 37.6 |
| 26.2 | 3.4 | 29.3 |
| 27.4 | 3.3 | 19.5 |
| 28.0 | 3.2 | 14.8 |
| 29.3 | 3.0 | 15.7 |

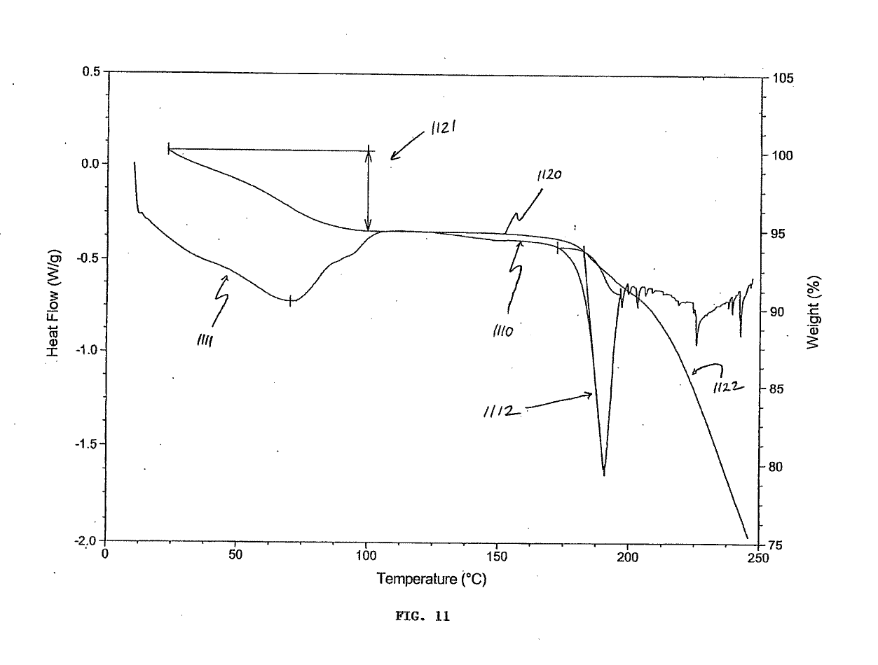

In another embodiment of the invention, Form H is characterized by a DSC profile 1110 shown in FIG. 11. The graph 1110 plots the heat flow from the sample as a function of temperature at a temperature scanning rate of 10° C./min. The graph 1110 is characterized by two endothermic transitions: the first transition 1111 has a maximum heat flow at 70° C., and the second transition 1112 has a maximum heat flow 191° C. The second transition 1112 begins at a temperature of about 182° C., and has an integrated heat output calculated to be 51.2 J/g of sample.

Form H of the Sulfate Salt is also characterized by a TGA profile 1120 shown in FIG. 11. The graph 1120 tracks the percent loss of weight of the sample as a function of temperature, the rate of change of the temperature being 10° C./min. The weight loss 1121 represents a loss of about 5.1% of the weight of the sample as the temperature is changed from room temperature to 100° C. Another weight loss 1122 begins at about the same temperature as the beginning of the endothermic transition 1112 of the DSC profile 1110. This weight loss 1122 corresponds with the melting and/or decomposition of the sample. Thus, the sample is characterized by a melting point/decomposition temperature of about 182° C. The temperatures corresponding to the DSC and TGA plots have an error of 3° C.

### Form D

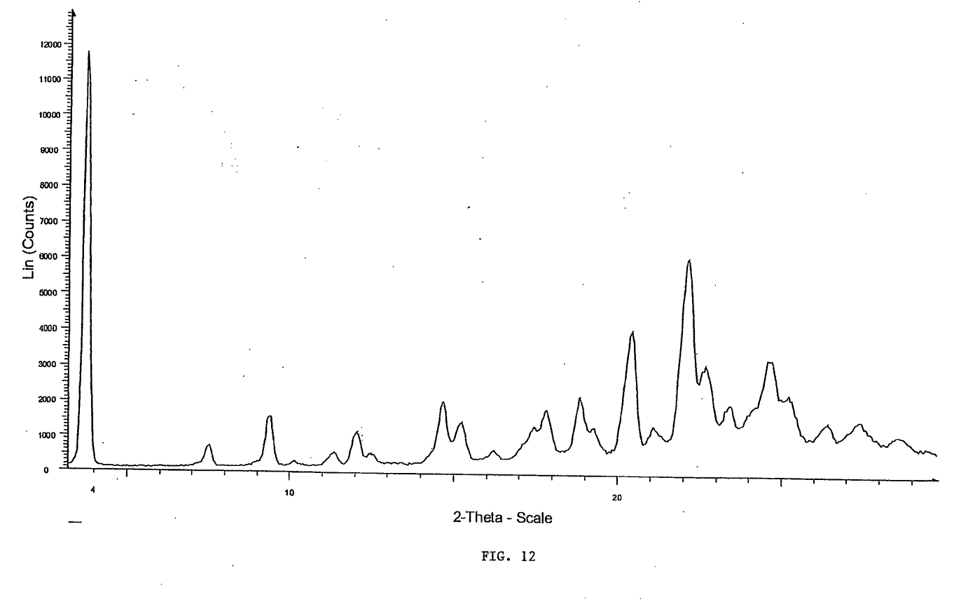

Form D is another polymorph of the Sulfate Salt. Form D is characterized by the XRPD profile shown in FIG. 12, created using CuKα radiation. The polymorph is also characterized by one, two, three, four, five, six, seven, eight, nine, ten, eleven, or twelve of the peaks shown in FIG. 12; a listing of some of the peaks is presented in Table 7. More particularly, the peaks are chosen from 3.7°, 10.1°, 12.0°, 15.2°, 16.2°, 17.8°, 22.7°, 23.4°, 24.7°, 26.4°, 27.4°, and 28.6°. The 2θ angles have an error of ±0.2°.

TABLE 7
XRPD Peaks from FIG. 12

| 2θ angle (°) | d value (Å) | Intensity % |
|---|---|---|
| 3.7 | 23.8 | 100 |
| 7.5 | 11.8 | 6.1 |
| 9.4 | 9.4 | 13.4 |
| 10.1 | 8.7 | 2.3 |
| 11.3 | 7.8 | 4.7 |
| 12.0 | 7.3 | 9.6 |
| 12.5 | 7.1 | 4.4 |
| 14.7 | 6.0 | 17.5 |
| 15.2 | 5.8 | 12.2 |
| 16.2 | 5.5 | 5.5 |
| 17.1 | 5.2 | 7.6 |
| 17.5 | 5.1 | 11.5 |
| 17.8 | 5.0 | 15.5 |
| 18.9 | 4.7 | 18.8 |
| 19.3 | 4.6 | 11.4 |
| 20.4 | 4.3 | 34.6 |
| 21.1 | 4.2 | 11.8 |
| 22.2 | 4.0 | 52.0 |
| 22.7 | 3.9 | 26.8 |
| 23.4 | 3.8 | 17.3 |
| 24.2 | 3.7 | 17.1 |
| 24.7 | 3.6 | 28.1 |
| 25.3 | 3.5 | 19.8 |
| 26.4 | 3.4 | 12.8 |
| 27.4 | 3.2 | 13.4 |
| 28.6 | 3.1 | 9.8 |

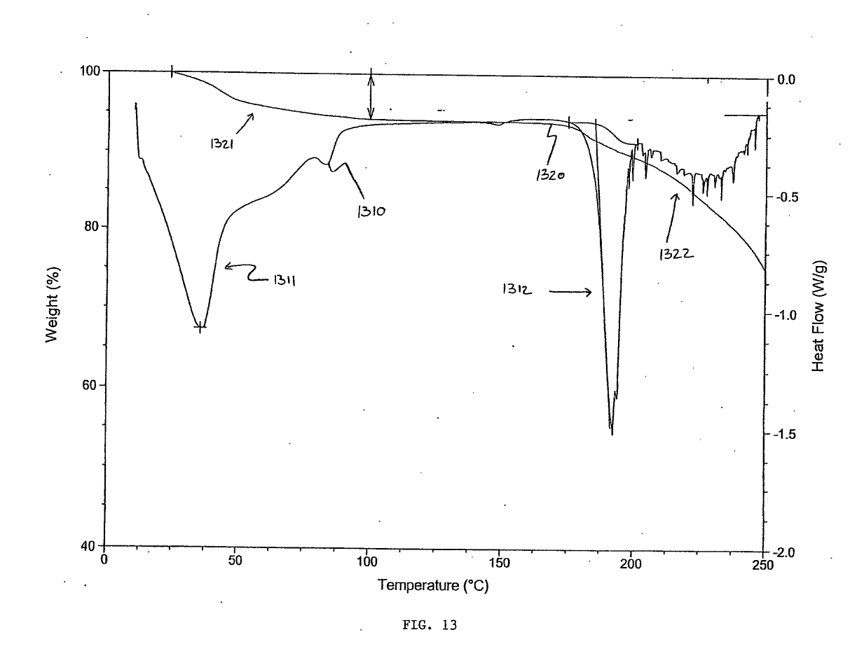

In another embodiment of the invention, Form D is characterized by a DSC profile 1310 shown in FIG. 13. The graph 1310 plots the heat flow from a sample of Form D as a function of temperature at a temperature scanning rate of 10° C./min. The graph 1310 is characterized by two endothermic transitions: the first transition 1311 has a maximum heat flow at 36° C., and the second transition 1312 has a maximum heat flow 193° C. The second transition 1312 begins at a temperature of about 185° C., and has an integrated heat output calculated to be 60.9 J/g of sample.

Form D is also characterized by a TGA profile 1320 shown in FIG. 13. The graph 1320 tracks the percent loss of weight of the sample as a function of temperature. The weight loss 1321 represents a loss of about 5.8% of the weight of the sample as the temperature is changed from room temperature to 100° C. Another weight loss 1322 begins at about the same temperature as the beginning of the endothermic transition 1312 of the DSC profile 1310. This weight loss 1322 corresponds with the melting and/or decomposition of the sample. Thus, the sample is characterized by a melting point/decomposition temperature of about 185° C. The temperatures of the DSC and TGA profiles have an error of ±3° C.

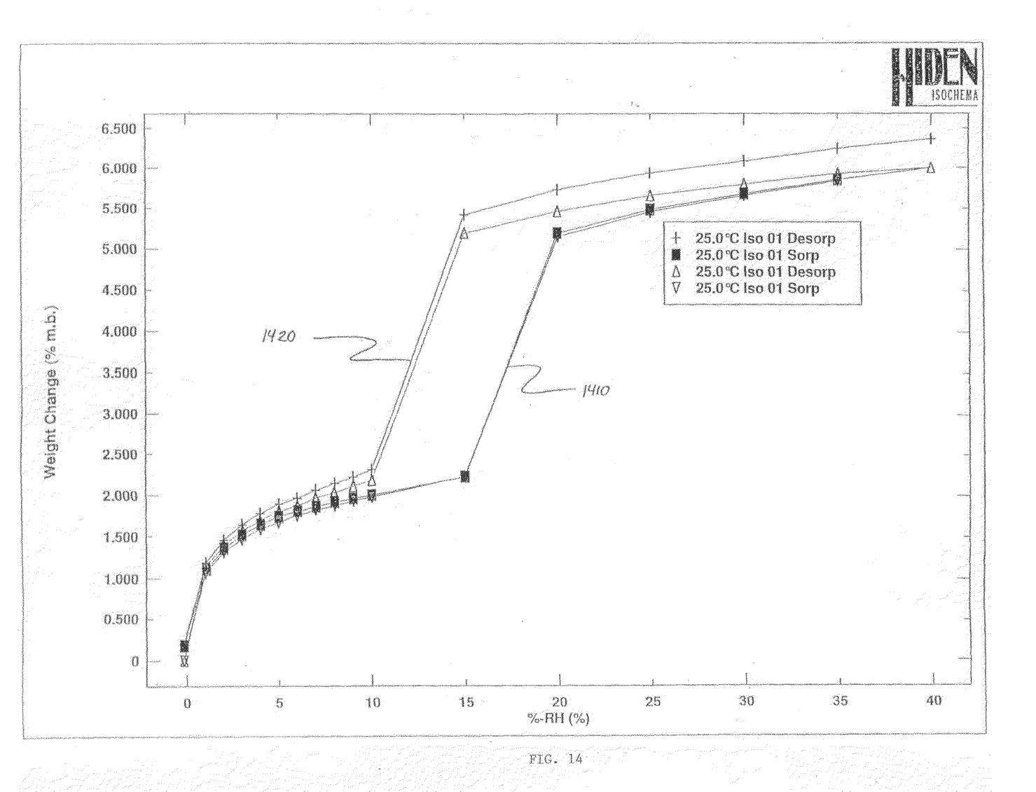

Form D is also characterized by the vapor sorption profiles shown in FIG. 14. The profiles 1410, 1420 show the change in weight of a sample of Form D, on an anhydrous basis, as the relative humidity of the environment is cycled between 0% and 40% at a temperature of 25° C. Each profile shows a transition weight change between about 2.0-2.5% and 5.0-5.5%. The vapor sorption profiles also show a hysteresis phenomena, with the weight change transitions for each profile occurring at a higher relative humidity relative to Form C (compare with FIG. 3).

Other embodiments of the invention are directed to a single crystalline form of the Sulfate Salt characterized by a combination of the aforementioned characteristics of any of the single crystalline forms discussed herein. The characterization may be by any combination of one or more of the XRPD, TGA, DSC, and water sorption/desorption measurements described for a particular polymorph. For example, the single crystalline form of the Sulfate Salt may be characterized by any combination of the XRPD results regarding the 2θ position of the major peaks in a XRPD scan; and/or any combination of one or more of the cell parameters derived from data obtained from a XRPD scan. The single crystalline form of the Sulfate Salt may also be characterized by TGA determinations of the weight loss associated with a sample over a designated temperature range; and/or the temperature at which a particular weight loss transition begins. DSC determinations of the temperature associated with the maximum heat flow during a heat flow transition and/or the temperature at which a sample begins to undergo a heat flow transition may also characterize the crystalline form. Weight change in a sample and/or change in sorption/desorption of water per molecule of anhydrous Sulfate Salt as determined by water sorption/desorption measurements over a range of relative humidity (e.g., 0% to 90%) may also characterize a single crystalline form of the Sulfate Salt.

Examples of combinations of single crystalline form characterizations using multiple analytical techniques include the 2θ location of at least one of the major peaks of a XRPD scan and the temperature associated with the maximum heat flow during one or more heat flow transitions observed by a corresponding DSC measurement; the 2θ location of at least one of the major peaks of a XRPD scan and one or more weight losses associated with a sample over a designated temperature range in a corresponding TGA measurement; the 2θ location of at least one of the major peaks of a XRPD scan, the temperature associated with the maximum heat flow during one or more heat flow transitions observed by a corresponding DSC measurement, and one or more weight losses associated with a sample over a designated temperature range in a corresponding TGA measurement; and the 2θ location of at least one of the major peaks of a XRPD scan, the temperature associated with the maximum heat flow during one or more heat flow transitions observed by a corresponding DSC measurement, one or more weight losses associated with a sample over a designated temperature range in a corresponding TGA measurement, and the change in sorption/desorption of water per molecule of anhydrous salt as determined by water sorption/desorption measurements over a range of relative humidity. As well, each of the aforementioned examples may replace the use of the 2θ location of at least one of the major peaks of a XRPD scan with one or more cell parameters of the single crystalline form.

The combinations of characterizations that are discussed above may be used to describe any of the polymorphs of the Sulfate Salt discussed herein (e.g., Form B, C, D, F, G, or H).

Embodiments of the invention are also directed toward a pharmaceutical composition comprising a pharmaceutically acceptable carrier or diluent; and a sulfate salt, or a single crystalline form of the sulfate salt, of 4-[6-methoxy-7-(3-piperidin-1-yl-propoxy)quinazolin-4-yl]piperazine-1-carboxylic acid (4-isopropoxyphenyl)-amide as described herein.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In a particular embodiment of the invention, a pharmaceutical composition comprises the Sulfate Salt and at least one of silicon dioxide, microcrystalline cellulose, pregelatinized starch, sodium stearyl fumarate, and sucrose. More particularly, the pharmaceutical composition comprises a crystalline form of the Sulfate Salt, preferably a single crystalline form and more preferably Form C, at a weight percent of about 40% to about 60% of the pharmaceutical composition, and includes at least one of silicon dioxide and microcrystalline cellulose. Even more particularly, an embodiment of the invention is directed to a pharmaceutical composition comprising about 50% of Form C of the Sulfate Salt, about 3% silicon dioxide (e.g., Cab-O-Sil M-5P, Cabot), about 10% pregelatinized starch (e.g., Spresss B820, GPC), about 2% sodium stearyl fumarate (e.g., Pruv, Penwest), and about 35% microcrystalline cellulose (e.g., Avicel PH-101, FMC Biopolymer).

In another particular embodiment of the invention, a pharmaceutical composition, as described herein, is prepared by granulating the composition. More particularly, the granulation is performed using a roller compactor (e.g., an Alexanderwerk WP 120×40 V) to granulate the composition to a form appropriate for capsule filling. The pressure exerted by a roller compactor, and the speed of the compactor, may be any that is suitable to achieve the desired granulation. In particular, pressures between about 20 bar and 100 bar may be used. In a particular embodiment of the invention the pharmaceutical composition comprising about 50% of Form C of the Sulfate Salt, about 3% silicon dioxide, about 10% pregelatinized starch, about 2% sodium stearyl fumarate, and about 35% microcrystalline cellulose, is roller compacted at a pressure between about 60 bar and about 100 bar at a roller speed of 3 revolutions per minute. In a related embodiment of the invention, a pharmaceutical composition is prepared in accord with the methods described in this paragraph.

In a preferred embodiment of the invention, a combination of the Sulfate Salt with silicon dioxide, microcrystalline cellulose, pregelatinized starch, sodium stearyl fumarate, and sucrose is granulated using a roller compactor (Alexanderwerk WP 120×40 V) to form a pharmaceutical composition suitable for capsule-filling. In a more preferred embodiment of the invention, the pharmaceutical composition comprises, by weight, about 50% of Form C of the Sulfate Salt, about 3% silicon dioxide (Cab-O-Sil M-5P, Cabot), about 10% pregelatinized starch (Spresss B820, GPC), about 2% sodium stearyl fumarate (Pruv, Penwest), and about 35% microcrystalline cellulose (Avicel PH-101, FMC Biopolymer). The mixture is roller compacted in the pressure range of 60 to 100 bar at a roller speed of about 3 revolutions per minute. The resulting product has a bulk density of 0.293 g/mL; a tapped density of 0.509 g/mL, as measured by a VanKel Tapped Density Tester employing 300 to 600 taps; and a Carr Index of 42.5%.

In other embodiments of the invention, an effective amount of the Sulfate Salt is administered to a subject in need of tyrosine kinase inhibition. The tyrosine kinase may be particularly the FLT-3 receptor tyrosine kinase. Inhibiting excessive tyrosine kinase activity may serve to treat a number of diseases involving cell survival, proliferation, and migration, including cardiovascular disease (e.g., arteriosclerosis and vascular reobstruction), cancer, glomerulosclerosis fibrotic diseases and inflammation, and general treatment of cell-proliferative diseases. In preferred embodiments of the invention, the disease is lung cancer, breast cancer, colorectal cancer, pancreatic cancer, or prostate cancer. In more preferred embodiments, the disease is glioma or leukemia (e.g., acute lymphocytic leukemia). In a still more preferred embodiment, the disease is acute myeloid leukemia. The Sulfate Salt may include a crystalline Sulfate Salt, or a single crystalline form of the Sulfate Salt, characterized by any of the Sulfate Salts described herein.

An “effective amount” refers to an amount effective to inhibit development of, or to alleviate the existing symptoms of, the subject being treated without including unacceptable side effects. Determination of the effective amount is well within the capability of those skilled in the art, especially in light of the detailed disclosure provided herein. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary within this range depending upon the dosage form employed, and the route of administration utilized. The exact formulation, route of administration, and dosage is chosen by the individual physician in view of the patient's condition. Dosage amount and interval can be adjusted individually to provide plasma levels of the active compound that are sufficient to maintain desired therapeutic effects.

In accord with preferred embodiments of the invention, the Sulfate Salts are formulated for pharmaceutical administration to a subject including mammals, and preferably human beings. Pharmaceutical compositions may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously.

Sterile injectable forms of compositions including the Sulfate Salt may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, isotonic sodium chloride solution, and dextrose. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Sulfate Salts consistent with embodiments of the invention may be orally administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate or sodium stearyl fumarate, are also typically added. For oral administration in a capsule form, useful diluents include lactose and starch (e.g., dried cornstarch or pregelatinized starch). Other useful excipients include colloidal silicon dioxide, microcrystalline cellulose, and sucrose. When aqueous suspensions are required for oral use, the active ingredient is combined with emulsifying and suspending agents. If desired, certain sweetening, flavoring or coloring agents may also be added.

Alternatively, the Sulfate Salt may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The Sulfate Salt may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the Sulfate Salt may be formulated in a suitable ointment containing the active component suspended or dissolved in one or more carriers. Carriers for topical administration of the Sulfate Salt include, but are not limited to, mineral oil, liquid petrolatum, white petrolatum, propylene glycol, polyoxyethylene, polyoxypropylene compound, emulsifying wax and water. Alternatively, the Sulfate Salt may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Some embodiments of the invention are directed toward methods of producing particular crystalline forms of the sulfate salt of 4-[6-methoxy-7-(3-piperidin-1-yl-propoxy)quinazolin-4-yl]piperazine-1-carboxylic acid (4-isopropoxyphenyl)-amide. As discussed herein, processes for making the Sulfate Salt in the prior art provide the Compound in a mixture of polymorphs, and in a material form with varying solubility.

In one particular embodiment of the invention is directed to a method for crystallizing Form C of the Sulfate Salt. A crude form of the Sulfate Salt may be obtained according to the procedures found in the published U.S. patent application bearing Ser. No. 10/041,160, filed Jan. 8, 2002; the entire contents of the published U.S. application is hereby incorporated by reference herein.

In step 1, crude Sulfate Salt is provided. The crude Sulfate Salt is dissolved in an approximately 3:1 mixture of denatured ethanol and water by volume. The total volume of the ethanol and water solvent mixture used is preferably sufficient to completely solubilize the crude Sulfate Salt when the mixture is heated to about 55° C. In one particular example, crude Sulfate Salt was charged with ethanol (9.375 liters/kilogram) and water (3.125 liters/kilogram).

In step 2, the mixture of step 1 is heated to about 55° C., subjected to a polish filtration, and collected in a clean vessel. The mixture is held at about 55° C. for a total time of less than about one hour, preferably for a period between about 10 minutes and one hour.

In step 3, the mixture of step 2 is held at that temperature for about 15 minutes. It is preferred that the mixture is held at about 55° C. for at least one minute and no longer than about one hour. More preferably, the mixture is held at about 55° C. for a period between about one and 30 minutes and most preferably, for a period between about 1 and 20 minutes.

In step 4, the mixture of step 3 is cooled to about 51° C. at the rate of about 6° C. per hour. It is preferred that the cooling rate is between about 5 and 7° C. per hour.

In other embodiments of the invention, the seeding of solutions utilizing the aforementioned method may further promote the formation of Form C of the Sulfate Salt. For example, after step 5, the mixture may be seeded with Form C crystals in an amount of about 0.5% by weight of the crude Sulfate Salt originally utilized in step 1. The seeded solution is held at 51° C. for 15 minutes, then cooled to 49° C. preferably at the rate of about 6° C. per hour. Subsequently, another seeding of Form C crystals, in an amount of about 0.2% by weight of the crude Sulfate originally utilized in step 1, may be added to the mixture to promote formation of the Form C polymorph preferentially. The solution is then cooled to 40° C. at a rate of 6° C. per hour. Cooling is then continued to 25° C. at a rate of 9° C. per hour. Finally, the mixture is cooled to about 0° C. to about 5° C. at a rate of 20° C. per hour. The mixture is centrifuged and the supernatant discarded. The crystals are washed with about 2 liters of ethanol per kilogram of crystals to be washed. The crystals are subsequently dried to yield the Form C polymorph of the Sulfate Salt.

A related embodiment of the invention is directed to a mixture for crystallizing a single crystalline form of the Sulfate Salt. The mixture comprises a sulfate salt of 4-[6-methoxy-7-(3-piperidin-1-yl-propoxy)quinazolin-4-yl]piperazine-1-carboxylic acid (4-isopropoxyphenyl)-amide; an organic solvent; and water. The mixture is preferably substantially free of non-Sulfate Salt solids (i.e., being substantially free of solids other than solid forms of the Sulfate Salt). In a particular embodiment, the organic solvent is ethanol. The mixture, including ethanol, is at a temperature above about 50° C. More particularly, the mixture may be at a temperature between about 40° C. and about 55° C. Also, the mixture may include crystals of Form C of the Sulfate Salt, the weight of the crystals being at least 0.1% by weight of the Sulfate Salt in the mixture. Also, a mixture can be in a single phase (e.g., liquid) or a multi-phase (e.g., solid and liquid).

In another embodiment of the invention, Form B of the Sulfate Salt is prepared by hydrating Form C of the Sulfate Salt.

In a first example, a sample of Form C of the Sulfate Salt is dissolved in water to form a solution. The solution is left standing, allowing Form B to crystallize directly from solution at room temperature over a period of hours.

In a second example, a slurry of Form C of the Sulfate Salt is mixed with water in about a 1:2 ratio by weight. The slurry is stirred for about 16 hours. Next, the supernatant was removed, and the solid dried in a vacuum oven at room temperature for about 2 hours. The resulting solid was analyzed using XRPD, DSC, and TGA; the analyses being consistent with a Form B of the Sulfate Salt.

In a third example, a sample of Form C of the sulfate salt was dissolved in water in about a 1:4 ratio by weight to form a solution. The solution was left standing for about 16 hours to form a precipitate. Removal of the supernatant and drying of the precipitate in a vacuum oven at room temperature for 2 hours yielded a solid consistent with the Form B of the Sulfate Salt as determined by XRPD, DSC, and TGA.

In related embodiments of the invention, Form B of the Sulfate Salt is prepared by hydrating Form C in a solution of water and another organic solvent. In one instance, a mixture of water and methanol, preferably in a volume ratio of about 3:1 to about 1:3, and Form C is prepared to yield Form B of the Sulfate Salt. Alternatively, mixtures of ethanol and water, preferably in a volume ratio of about 1:1, may be added to Form C to prepare a sample of Form B. Mixtures of water with one of isopropyl alcohol, 1-butanol, acetone, and tetrahydrofuran may also be utilized, preferably in a volume ratio of about 1:1.

Table 8 presents experimental results from dehydrating a mixture of 100 milligrams of Form C and the corresponding solvent water mixture to create a sample containing Form B.

TABLE 8
Solvent/Water Mixtures Added to Form C and
Dehydrated to Create Form B

| | Vol. of Solvent Added | Vol. of Water Added |
|---|---|---|
| Solvent | (μL) | (μL) |
| methanol | 600 | 200 |
| methanol | 400 | 400 |
| methanol | 200 | 600 |
| ethanol | 400 | 400 |
| isopropyl alcohol | 400 | 400 |
| 1-butanol | 400 | 400 |
| acetone | 400 | 400 |
| tetrahydrofuran | 400 | 400 |

In another embodiment of the invention, Form F of the Sulfate Salt is prepared by dehydrating Form B of the Sulfate Salt.

In one example, about a 10 to 30 mg sample of Form B of the Sulfate Salt was deposited in a platinum crucible and placed in a TGA furnace at 100° C. for about 30 minutes. Samples of the salt were analyzed immediately afterwards by XRPD to show a Form F structure. Repeating the analysis at regular intervals for a week result in no change in the observed pattern of the XRPD. Further experiments in which the Form B Sulfate Salt was placed in an isothermal TGA furnace for 30 minutes at temperatures varying from 60° C. to 160° C. all resulted in the formation of Form F of the Sulfate Salt.

In another example, a sample of Form B of the Sulfate Salt was dried in a vacuum oven at 40° C. overnight. The resulting solid is the Form F polymorph.

In another embodiment of the invention, Form F of the Sulfate Salt is prepared by concentrating a mixture of Form C in at least one solvent. The solvents that may be used include ethyl acetate, isopropyl acetate, tetrahydrofuran, ethanol, or acetone. In experiments performed by the applicants, five samples of Form F of the Sulfate Salt were successfully prepared by mixing 100 micrograms of Form C of the Sulfate Salt in 800 μL individually of each of the above-listed five solvents.

In another embodiment of the invention, Form G of the Sulfate Salt is prepared by dehydrating a sample of the Form C polymorph. In one particular example, a sample of Form C of the Sulfate Salt was heated in intervals of 10° C. from room temperature to 200° C.; a XRPD in situ was taken of the sample at each interval. The Form C sample dehydrated at about 60° C. to about 65° C. The sample has a XRPD spectra indicative of Form G for temperatures beginning from above 60° C. to the melting point of the sample. Further testing of the similar samples using DSC and TGA produced the same temperature range at which Form C transformed to Form G.

In another embodiment of the invention, Form H of the Sulfate Salt is prepared by preparing a slurry containing Form F of the Sulfate Salt in methanol. Subsequent removal of the methanol results in a solid of Form H.

In one example, about 1.3 grams of Form F of the Sulfate Salt was mixed with about 25 mLs of methanol. The slurry was stirred overnight. The remaining supernatant was removed, and the solid dried in a vacuum oven for about 1 hour. The resulting solid was analyzed using XRPD, showing results consistent with Form H.

In another example, about 50 mg of Form F of the Sulfate salt was dissolved in about 2 mLs of methanol. Heat was applied to slowly evaporate the solvent over a 72 hour time period. The resulting crystallized product had the structure of Form H as determined from XRPD analysis.

In another embodiment of the invention, Form D of the Sulfate Salt is prepared by drying a sample of Form H of the Sulfate Salt. In one example, a sample of Form H of the Sulfate Salt is dried at 0% relative humidity in a nitrogen stream to form a product. The product has the structure of Form D when exposed to a higher relative humidity environment. The Form H sample may also be dried with a nitrogen stream having a low, though non-zero, relative humidity.

Embodiments of this invention are useful for coating stent devices. Stents have been shown to reduce restenosis, but are thrombogenic. A strategy for reducing the thrombogenicity of stents is to coat, embed, adsorb or covalently attach a thrombin-inhibiting agent to the stent surface. The Sulfate Salt may be used for this purpose. Compounds of the invention may be attached to, or embedded within soluble and/or biodegradable polymers that are suitable for coating a stent. Examples of such polymers include polyvinylpyrrolidone, polyhydroxy-propylmethacrylamide-phenol, polyhydroxyethyl-aspartamide-phenol, or polyethyleneoxide-polylysine substituted with palmitoyl residues, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. See European Application 761 251, European Application 604,022, Canadian Patent 2,164,684 and PCT Published Applications WO 96/11668, WO 96/32143 and WO 96/38136.

### EXPERIMENTAL

X-ray powder diffraction patterns for the samples were acquired on a Bruker AXS C2 GADDS diffractometer using CuKα radiation (40 kV, 40 mA), an automated XYZ stage, a laser video microscope for auto-sample positioning, and a HiStar two-dimensional area detector. X-ray optics utilized a single Gael multilayer mirror coupled with a pinhole collimator of 0.3 mm.

Beam divergence, i.e., the effective size of the X-ray beam on the sample, was approximately 4 mm. A θ-θ continuous scan mode was employed with a sample-to-detector distance of 20 cm, giving an effective 2θ range of 3.2°-29.8°. A typical exposure time of a sample is 120 seconds.

Samples run under ambient conditions were prepared as flat plate specimens using powder as received without grinding. Approximately 1 mg to 2 mg of the sample was lightly pressed on a glass slide to obtain a flat surface. Samples run under non-ambient conditions were mounted on a silicon wafer with a heat conducting compound. The sample was then heated to the appropriate temperature at heating rate of about 20° C. per minute, and subsequently held isothermally for about one minute before data collection was initiated.

To obtain data for a single crystalline structure, XRPD was performed utilizing a Bruker-Nonius Kappa CCD diffractometer equipped with an Oxford Cryosystems Cryostream cooling device. Crystal structures are usually solved with either SIR-97 or SHELXS-97, and refined with SHELXL-97. Unless otherwise stated, hydrogen atoms are placed geometrically and allowed to refine with isotropic displacement parameters.

Differential scanning calorimetry (DSC) data was collected on a TA Instruments Q1000 differential scanning calorimeter equipped with a 50 position autosampler. The energy and temperature calibration standard was indium. Samples were heated at a rate of 10° C. per minute between 10° C. and 230° C. A nitrogen purge flowing at 30 mL per minute was maintained over the sample during a scan. Between 1 mg and 3 mg of sample was analyzed. All samples were crimped in a hermetically sealed aluminium pan.

Thermal gravimetric analysis (TGA) data was collected on a TA Instruments Q500 thermal gravimetric analyzer, calibrated with Nickel/Alumel and running at a scan rate of 10° C. per minute. A nitrogen purge flowing at 60 ml per minute was maintained over the sample during measurements. Typically 10 mg to 20 mg of sample was loaded onto a pre-tared platinum crucible.

Gravimetric vapor sorption (GVS) data was collected using a Hiden IGAsorp moisture sorption analyser from Hiden Isochem, Ltd., running CFRSorp software. Sample sizes were typically 10 mg. A moisture adsorption/desorption isotherm was recorded by subjecting samples to a series of relative humidity (RH) steps.

With respect to FIG. 3, the RH step profile is shown in Table 9; two scans yielding a complete measurement cycle. All samples were loaded/unloaded at typical room humidity and temperature (40% RH, 25° C.). All samples were analysed by XRPD post GVS analysis. The standard isotherm was performed at 25° C. at 10% RH intervals over a 0-90% RH range. From these measurements, the waters of hydration associated with each molecule of a polymorph may be determined.

TABLE 9
Humidity Step Profile for GVS Measurements

| | |
|---|---|
| Scan1 | Scan2 |

| Adsorption | Desorption | Adsorption |
|---|---|---|
| 40 | 85 | 10 |
| 50 | 75 | 20 |
| 60 | 65 | 30 |
| 70 | 45 | 40 |
| 80 | 35 | |
| 90 | 25 | |
| | 15 | |
| | 5 | |
| | 0 | |

While this invention has been particularly shown and described with references to embodiments thereof, it will be understood by those skilled in the art that various changes in form and details may be made therein without departing from the scope of the invention encompassed by the appended claims.

What is claimed is:

1. A sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being crystalline.

2. A sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being a single crystalline form.

3. A sulfate salt of 4-[6-methoxy-7-(3-piperidin-1-yl-propoxy)quinazolin-4-yl]piperazine-1-carboxylic acid (4-isopropoxyphenyl)-amide, at least 80% by weight of the sulfate salt being a single crystalline form.

4. A sulfate salt of 4-[6-methoxy-7-(3-piperidin-1-yl-propoxy)quinazolin-4-yl]piperazine-1-carboxylic acid (4-isopropoxyphenyl)-amide, at least 95% by weight of the sulfate salt being a single crystalline form.

5. The sulfate salt of claim 2, wherein the single crystalline form is Form C.

6. The sulfate salt of claim 2, wherein the single crystalline form is characterized by at least one x-ray powder diffraction peak at 2θ angles of 3.7°, 11.1°, 12.1°, 15.5°, 17.3°, 22.6°, 23.9°, 25.6°, and 29.0°.

7. The sulfate salt of claim 6, wherein the single crystalline form is characterized by the x-ray powder diffraction pattern of FIG. 1.

8. The sulfate salt of claim 2, wherein the single crystalline form is characterized by endothermic transitions observed at 59° C. ±3° C. and 190° C. ±3° C. when differential scanning calorimetry is performed on the single crystalline form using a scanning rate of 10° C./minute.

9. The sulfate salt of claim 2, wherein the single crystalline form has 1.8 to 2.0 waters of hydration per molecule of the sulfate salt.

10. The sulfate salt of claim 2, wherein the single crystalline form is characterized by a melting point of about 184° C. to 189° C. when heated at a rate of 10° C./minute.

11. The sulfate salt of claim 2, wherein the single crystalline form is Form B.

12. The sulfate salt of claim 2, wherein the single crystalline form is characterized by at least one x-ray powder diffraction peak at 2θ angles of 7.1°, 8.0°, 9.0°, 11.1°, 12.7°, 14.4°, 15.0°, 16.1°, 17.1°, 19.4°, 20.9°, 23.4°, and 24.4°.

13. The sulfate salt of claim 12, wherein the single crystalline form is characterized by the x-ray powder diffraction pattern of FIG. 4.

14. The sulfate salt of claim 2, wherein the single crystalline form is characterized by endothermic transitions observed at 50° C. ±3° C. and 216° C. ±3° C. when differential scanning calorimetry is performed on the single crystalline form using a scanning rate of 10° C./minute.

15. The sulfate salt of claim 2, wherein the single crystalline form has 6 to 7 water sites per molecule of the sulfate salt.

16. The sulfate salt of claim 2, wherein the single crystalline form is characterized by a melting point of 213° C. when heated at a rate of 10° C./minute.

17. The sulfate salt of claim 2, wherein the single crystalline form is Form F.

18. The sulfate salt of claim 2, wherein the single crystalline form is characterized by at least one x-ray powder diffraction peak at 2θ angles of 7.2°, 9.0°, 10.3°, 16.6°, 22.1°, 22.8°, 24.0°, 25.9°, 26.6°, and 27.9°.

19. The sulfate salt of claim 18, wherein the single crystalline form is characterized by the x-ray powder diffraction pattern of FIG. 6.

20. The sulfate salt of claim 2, wherein the single crystalline form has 0.4 to 0.6 waters of hydration per molecule of the sulfate salt.

21. The sulfate salt of claim 2, wherein the single crystalline form is Form G.

22. The sulfate salt of claim 2, wherein the single crystalline form is characterized by at least one x-ray powder diffraction peak at 2θ angles of 3.8°, 10.1°, 11.6°, 13.0°, 15.9°, 17.2°, 18.1°, 22.2°, 23.1°, and 23.4°.

23. A pharmaceutical composition comprising:
a pharmaceutically acceptable carrier or diluent; and
a sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being a single crystalline form.

24. A pharmaceutical composition comprising:
a pharmaceutically acceptable carrier or diluent; and
a sulfate salt of 4-[6-methoxy-7-(3-piperidin-1-yl-propoxy)quinazolin-4-yl]piperazine-1-carboxylic acid (4-isopropoxyphenyl)-amide, at least 80% by weight of the sulfate salt being a single crystalline form.

25. A pharmaceutical composition comprising:
a pharmaceutically acceptable carrier or diluent; and
a sulfate salt of 4-[6-methoxy-7-(3-piperidin-1-yl-propoxy)quinazolin-4-yl]piperazine-1-carboxylic acid (4-isopropoxyphenyl)-amide, at least 95% by weight of the sulfate salt being a single crystalline form.

26. The pharmaceutical composition of claim 23, wherein the single crystalline form is Form C.

27. The pharmaceutical composition of claim 23, wherein the single crystalline form has 1.8 to 2.0 waters of hydration per molecule of the sulfate salt.

28. The pharmaceutical composition of claim 23, wherein the single crystalline form is characterized by a melting point of 184° C. when heated at a rate of 10° C./minute.

29. The pharmaceutical composition of claim 23, wherein the single crystalline form is characterized by at least one x-ray powder diffraction peak at 2θ angles of 3.7°, 11.1°, 12.1°, 15.5°, 17.3°, 22.6°, 23.9°, 25.6°, and 29.0°.

30. The pharmaceutical composition of claim 23, wherein the single crystalline form is characterized by endothermic transitions observed at 59° C. ±3° C. and 190° C. ±3° C. when differential scanning calorimetry is performed on the single crystalline form using a scanning rate of 10° C./minute.

31. The pharmaceutical composition of any one of claims 26-30, wherein the pharmaceutically acceptable carrier is at least one of silicon dioxide, microcrystalline cellulose, pregelatinized starch, sodium stearyl fumarate, and sucrose.

32. The pharmaceutical composition of claim 31, wherein about 40% to about 60% by weight of the pharmaceutical composition is the sulfate salt.

33. The pharmaceutical composition of claim 23, wherein the single crystalline form is Form B.

34. The pharmaceutical composition of claim 23, wherein the single crystalline form has 6 to 7 water sites per molecule of the sulfate salt.

35. The pharmaceutical composition of claim 23, wherein the single crystalline form is characterized by a melting point of 213° C. when heated at a rate of 10° C./minute.

36. The pharmaceutical composition of claim 23, wherein the single crystalline form is characterized by at least one x-ray powder diffraction peak at 2θ angles of 7.1°, 8.0°, 9.0°, 11.1°, 12.7°, 14.4°, 15.0°, 16.1°, 17.1°, 19.4°, 20.9°, 23.4, and 24.4°.

37. The pharmaceutical composition of claim 23, wherein the single crystalline form is characterized by endothermic transitions observed at 50° C. ±3° C. and 216° C. ±3° C. when differential scanning calorimetry is performed on the single crystalline form using a scanning rate of 10° C./minute.

38. The pharmaceutical composition of claim 23, wherein the single crystalline form is Form F.

39. The pharmaceutical composition of claim 23, wherein the single crystalline form has 0.4 to 0.6 waters of hydration per molecule of the sulfate salt.

40. The pharmaceutical composition of claim 23, wherein the single crystalline form is characterized by a melting point of 212° C. when heated at a rate of 10° C./minute.

41. The pharmaceutical composition of claim 23, wherein the single crystalline form is characterized by at least one x-ray powder diffraction peak at 2θ angles of 7.2°, 9.0°, 10.3°, 16.6°, 22.1°, 22.8°, 24.0°, 25.9°, 26.6°, and 27.9°.

42. The pharmaceutical composition of claim 23, wherein the single crystalline form is Form G.

43. The pharmaceutical composition of claim 23, wherein the single crystalline form is characterized by at least one x-ray powder diffraction peak at 2θ angles of 3.8°, 10.1°, 11.6°, 13.0°, 15.9°, 17.2°, 18.1°, 22.2°, 23.1°, and 23.4°.

44. A method of treating a subject in need of tyrosine kinase inhibition comprising administering to the subject an effective amount of a sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being a single crystalline form.

45. The method of claim 44, wherein tyrosine kinase is FLT-3 receptor tyrosine kinase.

46. The method of claim 44, wherein the disease is lung cancer, breast cancer, colorectal cancer, pancreatic cancer, and prostate cancer.

47. The method of claim 44, wherein the single crystalline form is one of Form C, Form B, Form F, and Form G.

48. A method of treating a subject with glioma or acute lymphocytic leukemia comprising administering to the subject an effective amount of a sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being a single crystalline form.

49. The method of claim 48, wherein the single crystalline form is one of Form C, Form B, Form F, and Form G.

50. A method of treating a subject with acute myeloid leukemia comprising administering to the subject an effective amount of a sulfate salt of 4-[6-methoxy-7-(3-piperidin-1-yl-propoxy)quinazolin-4-yl]piperazine-1-carboxylic acid (4-isopropoxyphenyl)-amide, at least 60% by weight of the sulfate salt being a single crystalline form.

51. The method of claim 50, wherein the single crystalline form is one of Form C, Form B, Form F, and Form G.

52. A method for preparing a sulfate salt of 4-[6-methoxy-7-(3-piperidin-1-yl-propoxy)quinazolin-4-yl]piperazine-1-carboxylic acid (4-isopropoxyphenyl)-amide, comprising:
providing a pharmaceutical composition comprising a single crystalline form of the sulfate salt, and a pharmaceutically acceptable carrier or diluent; and
granulating the pharmaceutical composition.

53. The method of claim 52, wherein granulating includes roller compacting the pharmaceutical composition.

54. The method of claim 53, wherein the pharmaceutical composition further comprises at least one of silicon dioxide, microcrystalline cellulose, pregelatinized starch, sodium stearyl fumarate, and sucrose.

55. The method of claim 54, wherein roller compacting occurs using a pressure between about 60 bar to about 100 bar.

56. A pharmaceutical composition comprising a sulfate salt of 4-[6-methoxy-7-(3-piperidin-1-yl-propoxy)quinazolin-4-yl]piperazine-1-carboxylic acid (4-isopropoxyphenyl)-amide prepared in accordance with the method of claim 52.

57. A pharmaceutical composition comprising a sulfate salt of 4-[6-methoxy-7-(3-piperidin-1-yl-propoxy)quinazolin-4-yl]piperazine-1-carboxylic acid (4-isopropoxyphenyl)-amide prepared in accordance with the method of claim 55.
